(19) United States
(12) Patent Application Publication
(10) Pub. No.: US 2023/0414817 A1
(43) Pub. Date: Dec. 28, 2023

(54) SCENT PRODUCING ORNAMENTAL DEVICE

(71) Applicant: Burk Peters, Sarnia (CA)

(72) Inventor: Burk Peters, Sarnia (CA)

(21) Appl. No.: 17/808,151

(22) Filed: Jun. 22, 2022

(51) Int. Cl. A61L 9/12; A61L 9/012; A47G 35/00

(52) U.S. Cl. CPC A61L 9/12; A61L 9/012; A47G 35/00; A61L 2209/133; A61L 2209/15

(57) ABSTRACT

A scent producing ornamental device that is operable to provide a plurality of alternate olfactory experiences wherein the present invention includes a plurality of scent production members. The present invention includes a body wherein the body has a frame and is provided in a preferred embodiment in an annular shape. A plurality of lower ornamental elements are suspendedly secured to the body. The frame of the body includes a first scent production member, a second scent production member and a third scent production member. Each scent production member is configured to provide an olfactory experience utilizing alternate media. A fourth scent production member is releasably secured to the lower ornamental elements and is configured to provide an additional olfactory experience. The present invention is configured to be suspendedly mounted to a suitable support structure.

### FIELD OF THE INVENTION

The present invention relates generally to ornamental devices, more specifically but not by way of limitation, a dreamcatcher wherein the dreamcatcher is configured to provide a plurality of olfactory experiences delivered through alternate types of media and/or elements.

### BACKGROUND

The history of the dreamcatcher is dated back to an Ojibwe legend according to which the “spiderwebs” protective charms originate with Spider Woman known as Asibikaashi; who takes care of the children and the people on the land. As the Ojibwe Nation spread to the corners of North America it became difficult for Asibikaashi to reach all the children. So the mothers and grandmothers weave webs for the children, using willow hoops and sinew, or cordage made from plants. The purpose of these charms is apotrpaic and not explicitly connected with dreams. Dream catchers were adopted in the Pan-American Indian movement of the 1960s and 1970s and gained popularity as a widely marketed Native crafts items in the 1980s. While dreamcatchers continue to be used in a traditional manner in their communities and cultures of origin, they were also adopted into the Pan-Indian movement of the 1960s and 1970s as a symbol of unity among the various Native American cultures or a general symbol of identification with Native America cultures.

In some Native American cultures, a dreamcatcher is a handmade hoop, on which is woven a net or web It may also be decorated with sacred items such as certain feathers or beads. Traditionally, dreamcatchers are hung over a bed as protection. The tradition originated in the Anishinaabe culture as “the spider web charm” having a hoop with woven string or sinew meant to replicate a spider's web, used as a protective charm for infants. Dreamcatchers can be used as a shared symbol of hope and healing and are still presented to individuals as gifts. While many can be elaborate in appearance one drawback with current designs is the inability to provide impact on other senses besides sight. It is known in the art that certain smells produce various health benefits such as but not limited to reduction of stress.

Accordingly, there is a need for a dreamcatcher which may enable the activation of other senses beyond sight.

### SUMMARY OF THE INVENTION

It is the object of the present invention to provide a scent producing dreamcatcher that is configured to provide a plurality of scents wherein the present invention includes a body that is configured to be suspendedly mounted to a suitable support structure.

Another object of the present invention is to provide a hanging ornamental device that is operable to produce a plurality of different scents utilizing alternate delivery elements wherein the present invention includes a first scent production member.

A further object of the present invention is to provide a scent producing dreamcatcher that is configured to provide a plurality of scents wherein the present invention includes a second scent production member.

Still another object of the present invention is to provide a hanging ornamental device that is operable to produce a plurality of different scents utilizing alternate delivery elements wherein the first scent production member is configured to receive and store scented beads.

An additional object of the present invention is to provide a scent producing dreamcatcher that is configured to provide a plurality of scents wherein the second scent production member is configured to receive and retain scented oils.

Yet a further object of the present invention is to provide a hanging ornamental device that is operable to produce a plurality of different scents utilizing alternate delivery elements wherein the present invention includes a third scent production member.

Another object of the present invention is to provide a scent producing dreamcatcher that is configured to provide a plurality of scents wherein the third scent production member further includes a replaceable cartridge member.

Still another object of the present invention is to provide a hanging ornamental device that is operable to produce a plurality of different scents utilizing alternate delivery elements wherein the present invention further includes at least one portion of fabric that is impregnated with a scent and releasably secured to the body so as to be periodically replaced.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is front perspective view of the present invention.

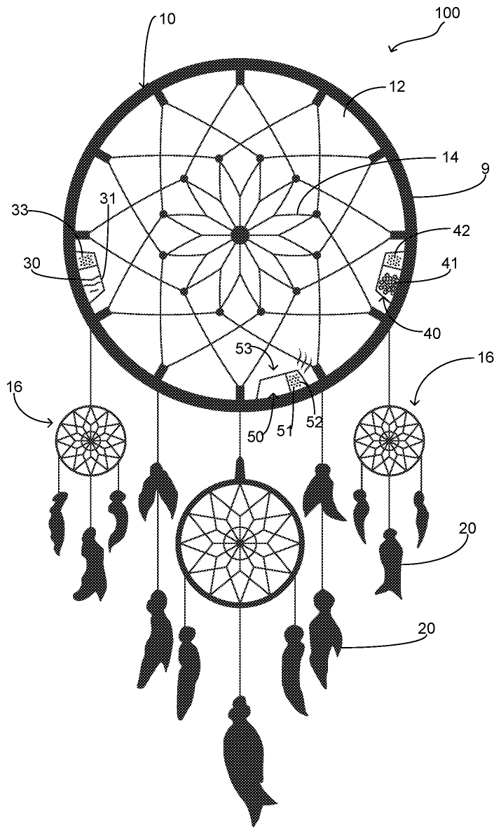

### DETAILED DESCRIPTION

Referring now to the drawings submitted herewith, wherein various elements depicted therein are not necessarily drawn to scale and wherein through the views and figures like elements are referenced with identical reference numerals, there is illustrated a scent producing ornamental device 100 constructed according to the principles of the present invention.

An embodiment of the present invention is discussed herein with reference to the FIGURE submitted herewith. Those skilled in the art will understand that the detailed description herein with respect to these FIGURE is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the FIGURE submitted as a part hereof, the scent producing ornamental device 100 includes a body 10. The body 10 is annular in shape and manufactured from a rigid material such as but not limited to wood. The body 10 includes a central void 12 wherein the central void 12 has ornamental elements 14 suspended therein. It should be understood within the scope of the present invention that the body 10 could be provided in alternate shapes and sizes. Furthermore, it should be understood within the scope of the present invention that the ornamental elements 14 could be provided in various designs and shapes. The scent producing ornamental device 100 further includes a plurality of lower ornamental elements 16. The lower ornamental elements 16 are suspendedly secured to the body 10 utilizing string or other suitable technique. It should be understood within the scope of the present invention that the scent producing ornamental device 100 could have no lower ornamental elements 16 or have a plurality thereof.

The lower ornamental elements 16 further include the fourth scent production member 20. The fourth scent production member 20 is releasably secured to the lower ornamental element 16 and is configured to emit a fragrance. The fourth scent production member 20 in a preferred embodiment is manufactured from fabric wherein the fabric has been impregnated with a scent emitting compound. Once suspended in the desired location, the fourth scent production member 20 is exposed to airflow which causes the release of the impregnated scent producing compound. Ensuing depletion of the scent production, the fourth scent production member 20 can be removed and replaced. It should be understood within the scope of the present invention that the fourth scent production member 20 could be impregnated with various scents employing alternate techniques. It should be further understood within the scope of the present invention that the scent producing ornamental device 100 could have various quantities of fourth scent production members 20.

The scent producing ornamental device 100 further includes a first scent production member 30. The first scent production member 30 is secured to the body 10 utilizing suitable techniques. The first scent production member 30 includes body 31 having an interior volume that is configured to receive and retain a liquid therein. The body 31 includes a releasably secured cap member 33 that includes a plurality of apertures that permits escape of a volatile liquid or oil disposed within the interior volume of the body 31. It should be understood within the scope of the present invention that the body 31 could be provided in alternate sizes and shapes. Furthermore, it is contemplated within the scope of the present invention that the first scent production member 30 could be integrated into the frame 9 of the body 10. The first scent production member 30 is operable to be periodically filled with an oil or volatile liquid so as to emit a desired fragrance.

The scent producing ornamental device 100 further includes a second scent production member 40. The second scent production member 40 is secured to the frame 9 utilizing suitable techniques. The second scent production member 40 includes a body 41 having a hollow interior volume configured to receive an retain elements such as but not limited to scented beads therein. The body 41 has releasably secured thereto a cap member 42 wherein the cap member 42 includes a plurality of apertures that enables the emission of fragrance from the material stored within the interior volume of the body 41. It should be understood within the scope of the present invention that the second scent production member 40 could be provided in alternate sizes and shapes.

The scent producing ornamental device 100 further includes a third scent production member 50. The third scent production member 50 is secured to the frame 9 of the body 10 utilizing suitable techniques. The third scent production member 50 includes a body 53 having a releasably secured cartridge 51 secured thereto. The cartridge 51 is further operably coupled to a cap member 52 wherein the cap member 52 includes a plurality of apertures that facilitate the emission of a scent. The cartridge 51 contains therein a volatile compound that is operable to emit a fragrance. The cartridge 51 is releasably secured to the body 53 and periodically replaced as the volatile compound therein becomes depleted. The cap member 52 assists in securing the cartridge 51 to the body 53. It should be understood within the scope of the present invention that the third scent production member 50 could be provided in alternate sizes and shapes. While a plurality o scent production members are present in the preferred embodiment of the scent producing ornamental device 100, it is contemplated within the scope of the present invention that the scent producing ornamental device 100 could have as few as one scent production member or more than four scent production members.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A scent producing ornamental device that is operable to provide an olfactory experience being configured with a plurality of scent production members wherein the scent producing ornamental device comprises:
a body, said body having a frame, said body further having a central void, said central void having at least one decorative element secured therein, said body configured to be suspendedly mounted to a support structure;
a first scent production member, said first scent production member being secured to said frame of said body, said first scent production member having a body wherein said body of said first scent production member has an interior volume configured to receive and retain a volatile liquid or oil, said first scent production member further including a cap member configured to permit emission of a gas therethrough; and
a plurality of lower ornamental elements, said plurality of lower ornamental elements being suspendedly mounted to said frame of said body, said plurality of ornamental elements having a fourth scent production member releasably secured thereto, said fourth scent production member being configured to emit a fragrance.

2. The scent producing ornamental device that is operable to provide an olfactory experience as recited in claim 1, and further including a second scent production member, wherein said second scent production member includes a body having an interior volume with a cap member secured to a top of the body of the second scent production member, said second scent production member operable to receive and retain scented beads therein.

3. The scent producing ornamental device that is operable to provide an olfactory experience as recited in claim 2, wherein said fourth scent production member is an impregnated fabric.

4. The scent producing ornamental device that is operable to provide an olfactory experience as recited in claim 3, and further including a third scent production member, said third scent production member including a body, said body of said third scent production member having a releasably secured cartridge, said cartridge containing a scent emitting compound.

5. The scent producing ornamental device that is operable to provide an olfactory experience as recited in claim 4, wherein said third scent production member further includes a cap member having a plurality of apertures wherein said cap member of said third scent production member is releasably secured to said body of said third scent production member.

6. The scent producing ornamental device that is operable to provide an olfactory experience as recited in claim 5, wherein the scent producing ornamental device is a dreamcatcher.

7. The scent producing ornamental device that is operable to provide an olfactory experience as recited in claim 6, wherein said body of the scent producing ornamental device is annular in shape.
